(19) United States
(12) Patent Application Publication
(10) Pub. No.: US 2020/0386098 A1
(43) Pub. Date: Dec. 10, 2020

(54) SOIL EXTRACTOR

(71) Applicant: AgriCORE LLC, New Haven, CT (US)

(72) Inventors: Ekaterina DANCHENKO, Geneva (CH); William Arthur SUSSMAN, Miller Place, NY (US); Pradhi AGGARWAL, Mumbai (IN); Francis KIGAWA, New York, NY (US)

(73) Assignee: AgriCORE LLC, New Haven, CT (US)

(21) Appl. No.: 16/894,220

(22) Filed: Jun. 5, 2020

Related U.S. Application Data
(60) Provisional application No. 62/858,577, filed on Jun. 7, 2019.

(51) Int. Cl. E21B 49/02; E21B 7/00; G01N 1/08; G01N 33/24

(52) U.S. Cl. CPC E21B 49/02; E21B 7/00; E02D 1/04; G01N 33/24; G01N 1/08

(57) ABSTRACT

A soil extractor is provided that allows a user to displace a drill bit along a drill pathway and collect a sample of soil at a desired depth without contaminating the sample with portions of the drill bit or any other portion of the soil extractor. Bushings protect the extractor itself, as well as the container/bucket from chipping or damage from the drill bit. This protection is what prevents contamination of the soil sample.

### CROSS-REFERENCE TO RELATED APPLICATIONS

This application claims the benefit of U.S. Provisional Patent Application 62/858,577, filed Jun. 7, 2019, entitled “Soil Extractor” the content of which is incorporated by reference herein in its entirety.

### FIELD OF THE DISCLOSURE

The present invention is directed to a soil sampling device that provides a measurable depth sample of soil without introducing impurities in the collected sample and without intensive labor.

### BACKGROUND OF THE DISCLOSURE

As disclosed herein, prior art sampling methods for soil include sampling with a shovel, sampling with a bucket with a hole in it (such as in the Collect-N-Go), and sampling with soil probes. These prior art methods are not easy to use at large scale, only the soil probe provides a measurable depth of sample, and the Collect-N-Go results in impurities in the sample due to the bucket chipping. No method provides a range of sampling depths and each method is very labor intensive.

Based on the foregoing, there is a need in the art for a device to sample soil in order to perform analyses on the soil. These analyses (carbon content analysis in particular) are used to evaluate and manage land. Carbon content analysis is important because it indicates the fertility of the soil and hence is integral for sustainable land management practices. There is a need for a technology that allows sampling to be performed quickly, easily, from a measurable range of depths, without the risk of contaminating the sample.

Wherefore it is an object of the present disclosure to overcome the above-mentioned shortcomings and drawbacks associated with conventional soil extractors.

### SUMMARY OF THE DISCLOSURE

There is disclosed herein a soil extractor assembly for removing soil from a ground. The soil extractor assembly uses a rotary drill provided by the user. The soil extractor includes a main shaft defined by a hollow cylinder extending from a lower end to an upper end.

A first bar is fixed directly via a first connector and a second connector at opposite ends. The first connector slidably mounts to the main shaft and the second connector supports a drill retainer. A first bushing and a second bushing are mounted to the second connector and the drill retainer surrounds the drill bit and defines an upper end of a drill pathway.

A second bar extends between a third connector and a fourth connector. A resizing bushing supports the second bar within the third connector and the fourth connector supports a drill bit chamber. The fourth connector and the drill bit chamber further define the drill pathway.

A removable container attaches to the drill bit chamber. The container has a drill aperture and the drill pathway continuing through the drill aperture.

A user moves the first bar along the main shaft towards the second bar and the drill retainer displaces within the second connector along the drill pathway in a first direction through the first bushing, the second connector, the second bushing, the drill aperture in the container, the drill bit chamber, and the fourth connector until the drill bit penetrates the soil. The user then moves the first bar along the main shaft away from the second bar and the drill retainer displaces along the drill pathway in an opposite second direction. The drill bit displaces a sample of the soil along the drill pathway in the second direction, the container retains the sample of soil, and the first bushing and the second bushing protect the container from contaminants generated by the drill.

In some embodiments, the first bar and the second bar are substantially parallel. In some embodiments, a number of apertures penetrate the main shaft and a stop engages one of the apertures at a desired depth. In some embodiments, the user adjusts the desired depth during use.

These aspects of the disclosure are not meant to be exclusive and other features, aspects, and advantages of the present disclosure will be readily apparent to those of ordinary skill in the art when read in conjunction with the following description, appended claims, and accompanying drawings.

### BRIEF DESCRIPTION OF THE DRAWINGS

The foregoing and other objects, features, and advantages of the disclosure will be apparent from the following description of particular embodiments of the disclosure, as illustrated in the accompanying drawings in which like reference characters refer to the same parts throughout the different views. The drawings are not necessarily to scale, emphasis instead being placed upon illustrating the principles of the disclosure.

FIG. 1 is a side view of a soil extractor according to the present disclosure with a container depicted in phantom;

FIG. 2A is a side view of a cap compatible with the soil extractor of FIG. 1;

FIG. 2B is a side view of a main shaft compatible with the soil extractor of FIG. 1;

FIG. 3 is a side view of a container compatible with the soil extractor of FIG. 1;

FIG. 4A is a side view of a first connector compatible with the soil extractor of FIG. 1;

FIG. 4B is a side view of a second connector compatible with the soil extractor of FIG. 1;

FIG. 4C is a side view of a first bar compatible with the soil extractor of FIG. 1;

FIG. 4D is a side view of a first bushing compatible with the soil extractor of FIG. 1;

FIG. 4E is a side view of a second bushing compatible with the soil extractor of FIG. 1;

FIG. 4F is a side view of a drill retainer compatible with the soil extractor of FIG. 1;

FIG. 5A is a side view of a third connector compatible with the soil extractor of FIG. 1;

FIG. 5B is a side view of a resizing bushing compatible with the soil extractor of FIG. 1;

FIG. 5C is a side view of a second bar compatible with the soil extractor of FIG. 1;

FIG. 5D is a side view of a drill bit chamber compatible with the soil extractor of FIG. 1; and

FIG. 5E is a side view of a fourth connector compatible with the soil extractor of FIG. 1;

### DETAILED DESCRIPTION OF THE DISCLOSURE

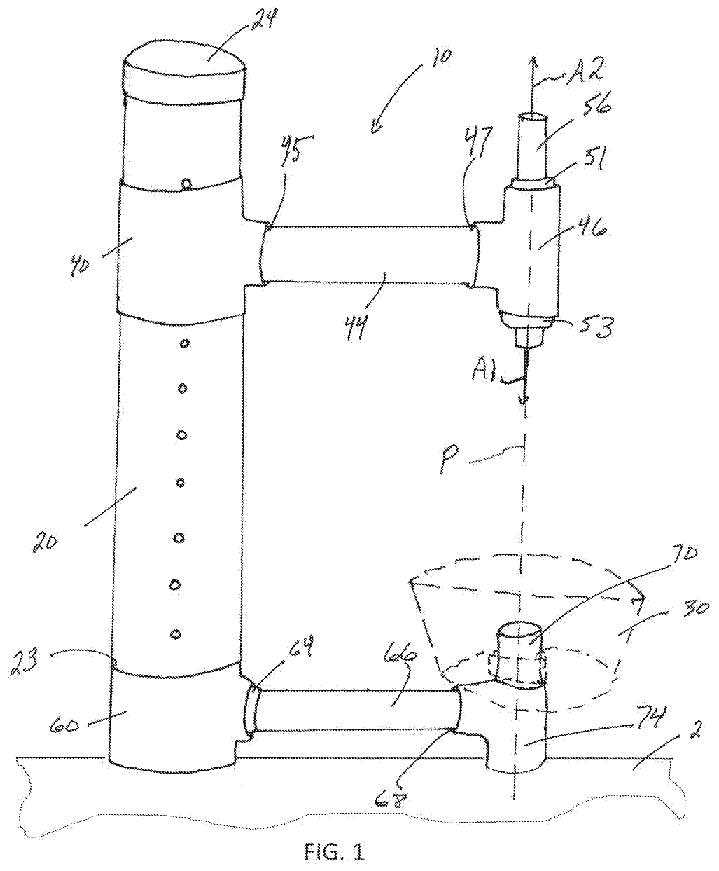

As shown in FIGS. 1-5E, a soil extractor is generally denoted by reference numeral 10. The soil extractor 10 includes a main shaft 20 attached to a moveable top bar or first bar 44 and a fixed bottom bar or second bar 66.

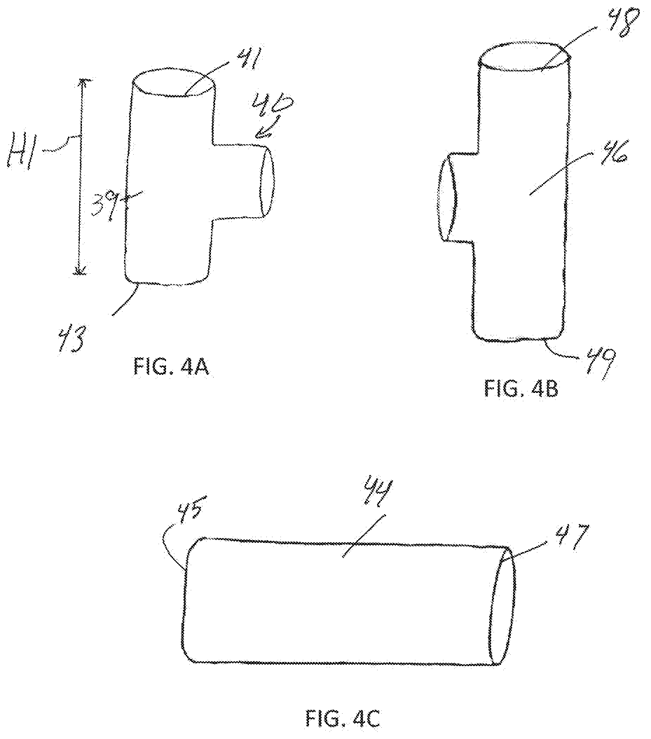

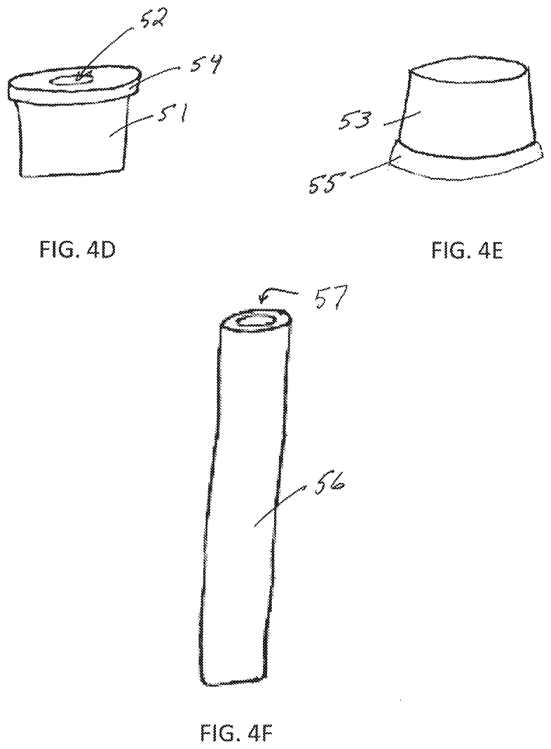

Referring to FIGS. 1 and 4A-4E, a first connector 40 is secured to a first end 45 of the first bar 44 and a second connector 46 at a second end 47 of the first bar 44. In the depicted embodiment, the first connector 40 surrounds and supports the first end 45 and the second connector surrounds and supports the second end 47, but other fixed connection means do not depart from the scope of the present disclosure. The first connector 40 supports the first bar 44 and the second connector 46 on the main shaft 20. Referring to FIG. 4A, the first connector 40 provides a cylindrical sidewall 39 extending from an upper edge 41 to a lower edge 43. The cylindrical sidewall 39 is complementary in shape to the main shaft 20 and has a vertical height H1 that provides sufficient support to maintain the first bar 44 and the second connector 46 in an upright alignment, as depicted in FIG. 1. The alignment of the first bar 44, the second bar 66, and the main shaft 20 are discussed in detail below.

Referring to FIGS. 4B and 4D-4F, the second connector 46 provides an upper aperture 48 that accommodates a first bushing 51 and a lower aperture 49 that accommodates a second bushing 53. In the depicted embodiment, the first bushing 51 and the second bushing 53 are structurally identical, but other arrangements do not depart from the scope of the disclosure. First bushing flange 54 axially supports the first bushing 51 on the upper aperture 48 and second bushing flange 55 axially supports the second bushing 53 on the lower aperture 49. A drill aperture 52 in each of the first bushing 51 and the second bushing 53 accommodates a drill retainer 56. The drill retainer 56 has a shank aperture 57 that surrounds and holds the shank surface of a drill bit (not depicted). The first bushing 51 seals the interface between the drill retainer 56 and the upper aperture 48 of the second connector 46 and the second bushing 53 seals the interface between the drill retainer 56 and the lower aperture 49 of the second connector 46. The first bushing 51 and the second bushing 53 accommodate the drill bit and the bushings prevent the extractor itself, as well as the bucket (often referred to as the container) from being chipped, which is what prevents contamination of samples when in use. The drill retainer 56 establishes a drill pathway P (as depicted in FIG. 1) that depicts the movement of the drill bit relative to the soil extractor 10 during use. The drill pathway P is discussed in detail below.

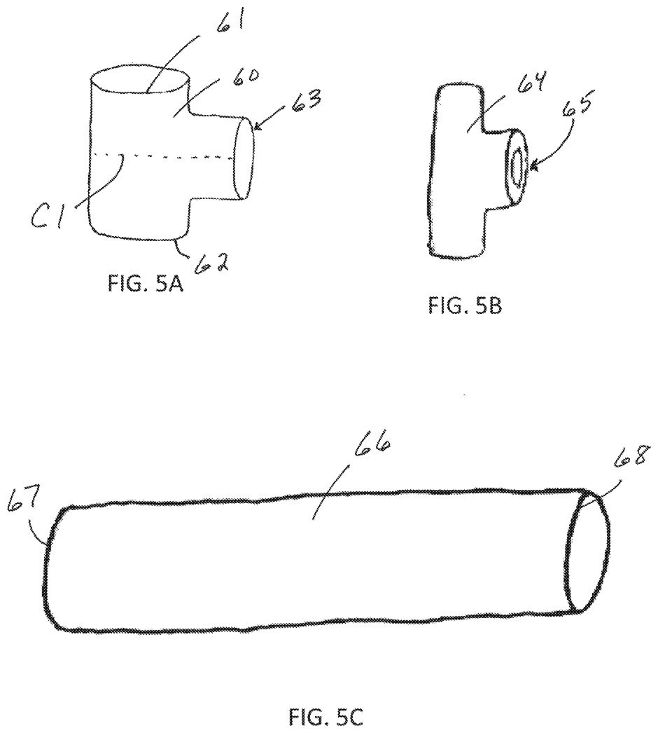

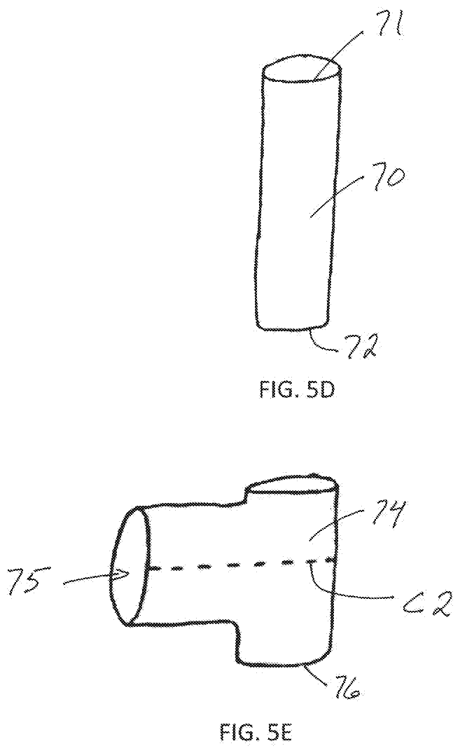

Referring to FIGS. 1 and 5A-5E, a resizing bushing 64 supports the second bar 66 within a third connector 60, fixedly connected to a lower end 23 of the main shaft 20. A fourth connector 74 is fixed to the second end 68 of the second bar 66. The fourth connector 74 receives a drill bit chamber 70 that completes the drill pathway P within the soil extractor 10. In the depicted embodiment, the lower edge 76 of fourth connector 74 is sharpened to allow the fourth connector 74 to sink into the ground 2.

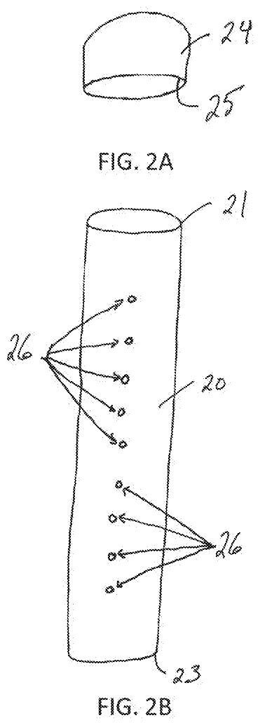

Referring to FIG. 1, a user operates the soil extractor 10 by a user pushing the first bar 44 in the first direction A1 until the drill retainer 56 passes through the drill bit chamber 70 and the drill bit penetrates the ground 2. As a result of this, the drill bit moves in the first direction A1 along drill pathway P. Holes 26 (depicted in detail in FIG. 2B) are drilled at specific distances along the main shaft 20. The holes 26 allow the user to stop the motion of the drill bit at specified depths, thereby ending the drill pathway P, by inserting dowels or stops (not depicted) into the holes 26. A user then pushes the first bar 44 in the second direction A2 along the drill pathway P. The drill bit lifts a sample of soil from the ground 2 and moves it along the drill pathway P within the drill bit chamber 70.

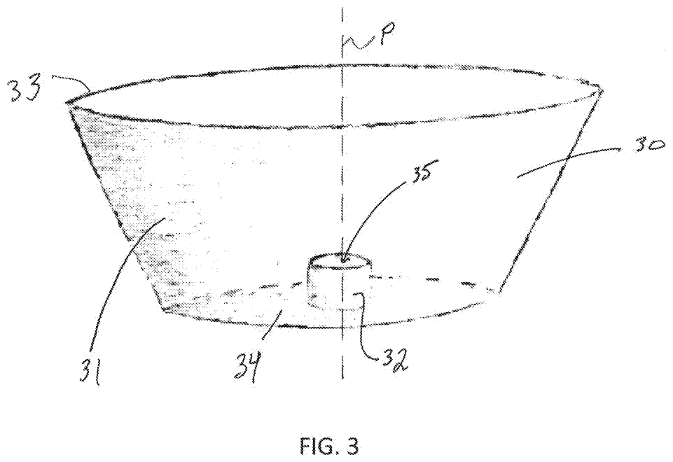

Referring to FIGS. 1 and 3, a bucket or container 30 sits on top of drill bit chamber 70. In the depicted embodiment, the container 30 has a sloped outer sidewall 31 and an inner sidewall 32. The inner sidewall 32 defines a container aperture 35 that allows the drill bit, drill retainer 56, and drill pathway P to pass therethrough. The container 30 collects the extracted soil (that gets ejected from the drill bit chamber 70 when the drill bit moves in the second direction A2 away from the ground 2). The size and shape of the inner sidewall 32 prevents soil from falling out of the container 30 through the container aperture 35. The container 30 is easily removable and replaceable. The design of the container 30 in conjunction with the drill pathway P and all related elements discussed above allow for an efficient collection of samples at a variety of depths without contamination.

The structure can also be manufactured and maintained for minimal cost. In some embodiments, the soil extractor 10 is made completely out of aluminum, except for the drill retainer 56 and the drill bit chamber 70 (which are both made out of steel) and the container 30 (which is made out of Polyethylene Terephthalate Glycol (“PETG”) plastic).

In at least one embodiment, as depicted in the present application and described in the following paragraph, the size of the elements relative to one another and the materials used allow the soil extractor 10 to be manufactured and repaired for a relatively low cost without hindering functionality of the soil extractor 10 or risking contamination of the soil sample. Referring to FIG. 2A, the cap 24 is 1.5 inches in diameter and made from Polyvinyl Chloride (“PVC”). In the embodiment depicted in FIG. 2B, the main shaft 20 is a hollow steel tube 3.5 inches in length and having a 1 inch width. In some cases, the main shaft is made of PVC. Nine (9) holes 26 are vertically aligned along the main shaft 20 and the holes 26 are substantially equidistantly spaced from one another. Referring to FIG. 3, the diameter of the container aperture 35 is 1 inch, the height of the inner wall 32 is 1.3 inches, the width of a bottom 34 is 7.9 inches, the vertical height of the outer sidewall 31 is 4 inches, and the width of an upper rim 33 is 11.8 inches in one direction and 3.2 inches in a perpendicular direction. Referring to FIG. 4A, the inner diameter of the cylindrical sidewall 39 is 1.5 inches and the first connector 40 is made from PVC. Referring to FIG. 4B, the second connector 46 is 1.5 inches in diameter and is made from PVC. Referring to FIG. 4C, the first bar 44 is a PVC pipe that is 4 inches long. Referring to FIGS. 4D and 4E, the first bushing 51 and the second bushing 53 each have a drill aperture 52 with a 0.75 inch inner diameter and the opposite end of each bushing 51, 53 has a 1.5 inch outer diameter. Referring to FIG. 4F, the shank aperture 57 of the drill retainer 56 has an inner diameter of 33/64 inches and is 3.5 inches long. Referring to FIG. 5A, the third connector 60 has an inner diameter of a third aperture 63 of 1.5 inches and a first center line C1 1.5 inches from a lower edge 62 of the third connector 60. Referring to FIG. 5B, the resizing bushing 64 is made from PVC, has an inner diameter of 0.75 inches of a conversion aperture 65, and has a height of 1.5 inches. Referring to FIG. 5C, the second bar 66 is hollow and made from steel. The second bar 66 is 4 inches in length and has a 1 inch outer diameter. Referring to FIG. 5D, the drill bit chamber 70 is a hollow steel tube with a 1 inch outer diameter and a 3.5 inch height. Referring to FIG. 5E, the fourth connector 74 has an outer diameter of a fourth aperture 75 of 1 inches and a second centerline C2 that is 1.5 inches from the lower edge 76. In the depicted embodiment, the first centerline C1 and the second centerline C2 are colinear.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

While various embodiments of the present invention have been described in detail, it is apparent that various modifications and alterations of those embodiments will occur to and be readily apparent to those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having,” and variations thereof herein, is meant to encompass the items listed thereafter and equivalents thereof as well as additional items while only the terms “consisting of” and “consisting only of” are to be construed in a limitative sense.

The foregoing description of the embodiments of the present disclosure has been presented for the purposes of illustration and description. It is not intended to be exhaustive or to limit the present disclosure to the precise form disclosed. Many modifications and variations are possible in light of this disclosure. It is intended that the scope of the present disclosure be limited not by this detailed description, but rather by the claims appended hereto.

A number of implementations have been described. Nevertheless, it will be understood that various modifications may be made without departing from the scope of the disclosure. Although operations are depicted in the drawings in a particular order, this should not be understood as requiring that such operations be performed in the particular order shown or in sequential order, or that all illustrated operations be performed, to achieve desirable results.

What is claimed is:

1. A soil extractor assembly for removing soil from a ground, the soil extractor assembly comprising:
a main shaft defined by a hollow cylinder extending from a lower end to an upper end;
a first bar having a first end fixed directly to a first connector and an opposite second end fixed directly to a second connector, the first connector slidably mounted to the main shaft, the second connector supporting a drill retainer disposed therein and having a first bushing mounted to an upper end of the second connector and a second bushing mounted to a lower end of the second connector, the drill retainer retaining the rotary drill and defining an upper end of a drill pathway;
a second bar extending from a third end received in a third connector to an opposite fourth end fixedly received in a fourth connector, a resizing bushing supports the third end of the second bar within the third connector, the fourth connector supporting a drill bit chamber, and the fourth connector and the drill bit chamber further defining a drill pathway therethrough; and
a container removeably attached to the drill bit chamber, the container defining a drill aperture and the drill pathway continuing therethrough,
wherein the first bushing and the second bushing protect the extractor and the container from contaminants generated by the drill.

2. The soil extractor assembly of claim 1, wherein a user displaces the first bar along the main shaft towards the second bar and the drill retainer displaces within the second connector along the drill pathway in a first direction through the first bushing, the second connector, the second bushing, the drill aperture in the container, the drill bit chamber, and the fourth connector until the rotary drill penetrates the soil.

3. The soil extractor assembly of claim 2, wherein a user then displaces the first bar along the main shaft away from the second bar and the drill retainer displaces along the drill pathway in an opposite second direction.

4. The soil extractor assembly of claim 1, wherein the first bar and the second bar are substantially parallel.

5. The soil extractor assembly of claim 1, further comprising a plurality of apertures penetrating the hollow cylinder and a stop retained in one of the plurality of apertures at a desired depth.

6. The soil extractor assembly of claim 5, wherein the user adjusts the desired depth during use.

7. A method for removing soil from a ground using a soil extractor assembly, the method comprising:
moving a first bar in a first direction towards a second bar along a main shaft of the soil extractor assembly, the first bar fixedly connected and moving with a drill retainer, a first bushing, a second bushing, and a drill bit all disposed within a second connector attached to the first bar;
moving the drill retainer and the drill bit in the first direction through a drill aperture in a container and then through a drill bit chamber attached to a second bar by a fourth connector, the second bar fixedly attached to the main shaft by a third connector and a resizing bushing contained therein;
penetrating the ground with the drill bit;
collecting a sample of soil with the drill bit;
moving the drill retainer and the drill bit in a second direction opposite the first direction through the fourth connector, the drill bit chamber, and the drill aperture; and
depositing the sample of soil in the container;
wherein a drill pathway is defined by the movement of the drill bit in the first direction and the second direction and
wherein the first bushing and the second bushing protect the extractor and the container from contaminants generated by the drill.
